(19) United States
(12) Patent Application Publication
(10) Pub. No.: US 2005/0079497 A1
(43) Pub. Date: Apr. 14, 2005

(54) Multiple oligonucleotides per gene for use in gene arrays

(75) Inventors: Simon H Sims, Houston, TX (US); T.S Ramasubramanian, The Woodlands, TX (US); Paul Appeddu, Houston, TX (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Sigma-Aldrich Co.

(21) Appl. No.: 10/494,220

(22) PCT Filed: Oct. 25, 2002
(86) PCT No.: PCT/US02/34554

Related U.S. Application Data
(60) Provisional application No. 60/345,884, filed on Oct. 27, 2001.

(51) Int. Cl. C12Q 1/68

(52) U.S. Cl. 435/6

(57) ABSTRACT

This disclosure includes an inventive method for nucleic acid array design, in which the array contains individual nucleic acid sequences or portions thereof, and in which the method comprises: (a) providing at least two discrete oligos per nucleic acid sequence or portions thereof; (b) printing the array with the oligos; and (c) using the array in genetic analysis. In a preferred embodiment, two or three oligos are advantageously provided per target nucleic acid sequence or portions thereof on the array. This disclosure further includes an inventive method for genetic analysis, in which the method comprises: (a) generating labeled nucleic acids from a sample nucleic acid population using probe matched target primers; (b) hybridizing the labeled nucleic acids to an array; and (c) analyzing the array. This disclosure further includes an inventive kit having component parts capable of being used in combination for testing genetic material for the presence or absence of predetermined nucleic acid sequences, the kit comprising the combination of: an array containing at least one discrete oligo per nucleic acid sequence or portions thereof, and at least two probe matched target primers.

This application claims the benefit of U.S. Provisional Application No. 60/345,884 filed on Oct. 27, 2001.

### TECHNICAL FIELD OF THE INVENTION

This application relates generally to gene arrays, and more specifically to improvements in the design and use of such gene arrays, such as the use of multiple oligonucleotides probes per gene and the use of probe matched target primers.

### BACKGROUND OF THE INVENTION

This disclosure occasionally uses the following acronyms:
mRNA=messenger ribose nucleic acid
cDNA=complementary DNA
oligo=oligonucleotide
rRNA=ribosomal RNA
tRNA=transfer RNA
snRNA=small nuclear RNA

Nucleotide arrays to date typically use Polymerase Chain Reaction (PCR) products as the material printed at each discrete spot within the array. Typically, one PCR product per target sequence is printed per spot and the size of the PCR products typically range from about one hundred bases to greater than two thousand bases. Arrays generated in this manner may be used in gene expression studies, in which a labeled cDNA is generated with the use of oligo dT primers or with a set of random primers, such as hexamers to decamers. The use of oligo dT primers results in the generation of cDNA molecules which are complementary to poly-adenylated RNA, but will typically not create cDNA molecules from any other type of RNA. The use of random primers, on the other hand, will typically result in the generation of cDNA molecules matching the positions within any form of RNA to which the primers are complementary, such as rRNA, TRNA, snRNA, as well as mRNA. cDNAs may be labeled during their generation using any number of mechanisms that will allow detection. Many of these methods involve the incorporation of fluorescently or radioactively labeled nucleotides. The resulting labeled cDNAs targets may then be hybridized to an array of immobilized probes for subsequent visualization or detection to determine which and how many of the probes are complementary to sequences with in the sample of cDNAs.

One of the most commonly encountered problems in using such an array is the resulting background or non-specific hybridization. This problem occurs as a result of the presence of regions of sequence cross-homology between different genes or polynucleotide sequences, such that there may be a stretch of any number of bases in one or more of the immobilized probes that show significant similarities between any one, two, three or more of the labeled sample sequences. This means that the sequences in the labeled target corresponding to a cDNA for a particular gene or region of interest may cross-hybridize to regions present in one or more probes resulting in a potential non-specific background signal. In such a situation, the gene probe of interest would display a very strong signal, as more of the cDNA would hybridize to this complimentary PCR generated probe. However, the other probe sequence(s) containing only a slight homology to the target may bind stably enough to result in detectable signals, thereby creating background or false positive signals indicating the presence of the sequence of interest.

These prior art issues are illustrated in FIG. 1. In FIG. 1A, two gene probes are represented such that Gene 1, the gene of interest, contains a region of cross-homology with Gene 2, the second gene. FIG. 1B depicts the generation of a labeled cDNA, where “X”s throughout denote labeled nucleotides. FIG. 1C depicts the results that would be expected from the use of a PCR probe array in which labeled target cDNAs, were created from a sample containing Gene 1 but not containing Gene 2. As shown, when the labeled cDNAs are hybridized to the array, the sample containing Gene 1 will correctly indicate the presence of Gene 1, while the sample will also falsely indicate the presence of Gene 2.

Another type of conventional array in use today implements oligonucleotides (“oligo”) probe instead of the PCR generated probes discussed above. FIG. 2 illustrates the use for such an oligonucleotide based array. FIG. 2A diagrams the general structure of an mRNA molecule with a 5′ untranslated region, a protein coding region, a 3′ untranslated region, and a poly-A tail. Typically, labeling reactions that generate cDNA targets use mRNA as a template and an oligo dT primer for initiating the reaction (in eukaryotes) or total RNA and random hexamers (prokaryotes). These allow reverse transcriptase to generate a cDNA target while incorporating labeled nucleotides, as depicted by the hatched line in FIG. 2B. Utilizing an oligonucleotide array to detect a particular cDNA requires the design of a sense stand oligonucleotide, depicted by the oscillating line in FIG. 2B, to be complimentary to the anti-sense, labeled cDNA target. In the typical assay, the labeled cDNA target hybridizes to its complementary immobilized oligonucleotide probe printed within the array to provide a detectable signal.

Typically, oligonucleotide probe design involves the use of software algorithms that select an optimal hybridization probe based on a number of different criteria, such as optimum melting temperature (TM) for the hybridization conditions to be used and the desired length of the oligonucleotide probe. In addition, oligonucleotide design attempts to minimize the potential secondary structures a molecule might contain, such as hairpin structures and dimmers between probes, with the goal being to maximize availability of the resulting probe for hybridization. A principal reason for the invention and use of oligonucleotide based arrays is that they may provide much greater specificity than their PCR based counter-parts. Oligonucleotide probes may be designed with the key parameter being the uniqueness of the oligonucleotide sequence in order to decrease the potential for cross-homology with other target sequences, as discussed in relation to a PCR based probe array. Therefore, signals generated using a distinctively designed oligonucleotide probe should be very specific for their targets resulting in fewer false positive results, whereas false positives can be quite common in PCR product based arrays.

While conventional oligonucleotide probe based arrays have the potential for much greater specificity, they also have an increased probability of generating false negatives as compared to PCR probe based arrays. FIGS. 2C and 2D illustrate two potential mechanisms through which such false negatives may occur in the prior art. In FIG. 2C, the oligonucleotide selected is located towards the 5′ end of the gene, possibly because it was the best location in relation to the uniqueness of region or had the least potential secondary structure and the most ideal hybridization characteristics. However, the use of oligo-dT primers the reverse transcriptase reaction to generate the labeled cDNA target, depicted by the hatched line, may not result in sufficient extension to produce that part of the sequence that would be complementary to the selected oligonucleotide probe. Therefore, the labeled target would not produce a signal within the array in spite of the fact that the sequence of interest was present in the sample. Alternatively, as shown in FIG. 2D, the possibility of secondary structures within the labeled cDNA target is illustrated, depicted by the hatched line, creating a false negative outcome. The cDNA may contain a hairpin loop, masking the sequence which is complementary to the oligonucleotide probe, thus making this area of the target unavailable for hybridization. This will result in no signal at that oligonucleotide probe despite the presence of its target sequence. FIG. 2, in general, shows examples as to why a signal might not be detected using an oligonucleotide probe based array, however, there may be other reasons which could result in the same outcome.

Another known method of array production is through the use of a photolithographic process. The photolithography process enables the synthesis of short oligonucleotides directly onto the surface of a substrate. However, due to inefficiencies of the chemical synthesis only relatively short oligonucleotides, probably up to about 25 bases, can be effectively synthesized by the process. In addition, the process requires the use of photolithographic masks which may take extensive periods of time to produce, decreasing the ease with which probe sequences can be changed.

FIG. 3 illustrates an overview of a typical use for such a photolithographic manufactured array. In FIG. 3A, an mRNA, is shown in parallel with oligonucleotides designed to be complementary to an antisense cDNA that are either exact matches, depicted as plain oscillating lines, or oligonucleotides designed to have a one base mismatch near the center of the molecule, depicted as oscillating lines containing an “X” thought their mid-point. Multiple oligonucleotides are used per target sequence in order to: (1) maximize the chance of detecting the target sequence without having to go back and redesign the masks to generate another array; and (2) to increase the specificity of the assay in light of the fact that the relatively short oligonucleotides have a greater chance of displaying cross-homology to non-target sequences. FIG. 3B diagrams the typical topography of the array with respect to probe sequences directed toward a given target, wherein each is represented by a discrete spot within the array such that there would be one row of exactly matched oligonucleotides and another row of mismatched oligonucleotides to act as a controls for non-specific binding. Therefore, theoretically, if the sequence of interest is present within the labeled sample, as depicted in FIG. 3C, the oligonucleotides of the exact matched probes will be positive for binding and the oligonucleotides of the mismatched probes will be negative. Part D depicts the typical results of such an assay. The exact matched row shows relatively strong signals for the particular sequence of interest and the mismatched probes may show no signal or a partial signal. The lack of signal from matched probes and the presence of signals from mismatched probes, necessitates the use of complex algorithms to compile and interpret the experimental results in order to statistically verify the presence of the targeted sequence.

There have been some recent attempts to address the need for increased specificity and accuracy. For example, U.S. Pat. No. 6,306,643 BI discloses arrays of polynucleotide probes bound to a support having at least one pooled position. A key aspect of the disclosure is the use of three locations within the array per target sequence with one location containing probes 1 and 2, a second location will contain probe 1 alone and a third location would contain probe 2 alone. This approach is directed, in part, toward the premise that two different probes in a pool of mixture of probes can simultaneously hybridize the different segments of the same target molecule in a cooperative manner, such that the binding of a target to a pool of two mixed probes is greater than the sum of binding of the targets to the same two probes separated into individual locations within the array, thereby increasing the potential sensitivity. Another reference that seeks to take advantage of cooperative binding to a given spot through the use of multiple probes is U.S. Patent Application No.: 20010055760. This reference, much like the U.S. Pat. No. 6,306,643 reference, discloses the use of multiple oligonucleotide probes capable of hybridizing, in a cooperative fashion, to distinct or different regions of a target sequence immobilized in a discrete spot within an array.

Therefore, there exists a need in the art for a nucleotide array system that increases specificity, while concomitantly reducing the need for utilizing multiple locations within the array to ensure the identification of targeted sequences.

### SUMMARY

These and other needs in the art are addressed by an inventive method for nucleic acid array design, in which the array contains individual nucleic acid sequences or portions thereof, and in which the method comprises: (a) providing at least two discrete oligos per nucleic acid sequence or portions thereof; (b) printing the array with the oligos; and (c) using the array in genetic analysis.

In a preferred embodiment, three oligos are advantageously provided per nucleic acid sequence or portions thereof on the array.

A technical advantage of this inventive method is that in providing at least two oligos per nucleic acid sequence or portions thereof, multiple oligo sequences complementary to the target are now located within a discrete spot on the array. This will be appreciated to increase the chances of obtaining a true positive signal (and/or decrease the chances of a false negative signal) in using the array to identify the presence of a predetermined nucleic acid sequence.

The above-described needs in the art are further addressed by an inventive method for genetic analysis, in which the method comprises: (a) generating labeled nucleic acids from a sample nucleic acid population using probe matched target primers; (b) hybridizing the labeled nucleic acids to an array; and (c) analyzing the array.

A technical advantage of this second inventive method is that by using probe matched target primers, the probability is increased of generating a labeled target that will specifically bind to the matched probe on the array.

The above-described needs in the art are yet further addressed by an inventive kit having component parts capable of being used in combination for testing genetic material for the presence of predetermined nucleic acid sequences, in which the kit comprises the combination of: an array containing at least one discrete oligo per nucleic acid sequence or portions thereof, and at least two probe matched target primers.

A technical advantage of this inventive kit is that it combines the materials required to perform the above described inventive methods into a convenient and commercially attractive package. Each kit enables a user to perform the inventive methods on separate nucleic acid identification assays, as desired.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates conventional PCR array methodology and includes FIGS. 1A, 1B and 1C;

FIG. 1A illustrates the possibility of cross homology in conventional PCR arrays;

FIG. 1B illustrates generation of labeled cDNA targets in conventional PCR arrays;

FIG. 1C illustrates background signal resulting from cDNA target hybridizing with PCR probe in conventional PCR arrays;

FIG. 2 illustrates conventional oligo probe based array methodology and includes FIGS. 2A, 2B, 2C and 2D;

FIG. 2A illustrates the structure of mRNA;

FIG. 2B illustrates oligonucleotide probe hybridized to target cDNA generating a signal in conventional oligo arrays;

FIG. 2C illustrates an oligonucleotide probe used in hybridization to cDNA target without generating a signal in conventional oligo arrays;

FIG. 2D illustrates where, in oligo arrays, an oligonucleotide probe may not anneal to hairpin cDNA target;

FIG. 3 illustrates conventional photolithographic array methodology and includes FIGS. 3A, 3B, 3C and 3D;

FIG. 3A illustrates multiple short oligonucleotide probes used to detect one gene, as used in conventional photolithographic arrays;

FIG. 3B illustrates a conventional photolithographic oligonucleotide probes on an array;

FIG. 3C illustrates hybridized labeled cDNA targets;

FIG. 3D illustrates signal strength between matched and mis-matched probes hybridized to target in conventional photolithographic arrays;

FIG. 4 illustrates the inventive method of multiple oligos per gene with further reference to FIGS. 4A, 4B, 4C and 4D;

FIG. 4A illustrates that only if cDNA extends to position of oligonucleotide probes, are targets detected;

FIG. 4B illustrates secondary structure effects probe and target binding;

FIG. 4C illustrates turnover of genetic material affects signals;

FIG. 4D illustrates differential gene expression in human cells depicted with probes;

FIG. 5 illustrates the inventive method of probe matched target primers (PMTPs) with further reference to FIGS. 5A, 5B and 5C;

FIG. 5A illustrates probe target labeling primers;

FIG. 5B illustrates post heat shock and control target cDNAs generated with different cDNA primers and hybridized to oligo probes;

FIG. 5C illustrates the comparative actions cDNA generated with oligo dT primers vs. cDNA probe matched primers hybridized to oligos on microarray;

FIG. 6 illustrates an embodiment of the PMTP invention sued in bacterium strain typing with further reference to FIGS. 6A and 6B;

FIG. 6A illustrates such bacterium strain typing;

FIG. 6B illustrates labeled DNA hybridized to matched probe oligonucleotide array in such bacterium strain typing;

FIG. 7 illustrates an embodiment of the PMTP invention used in differential RNA degradation analysis; and

FIG. 8 illustrates an aspect of the invention in kit form.

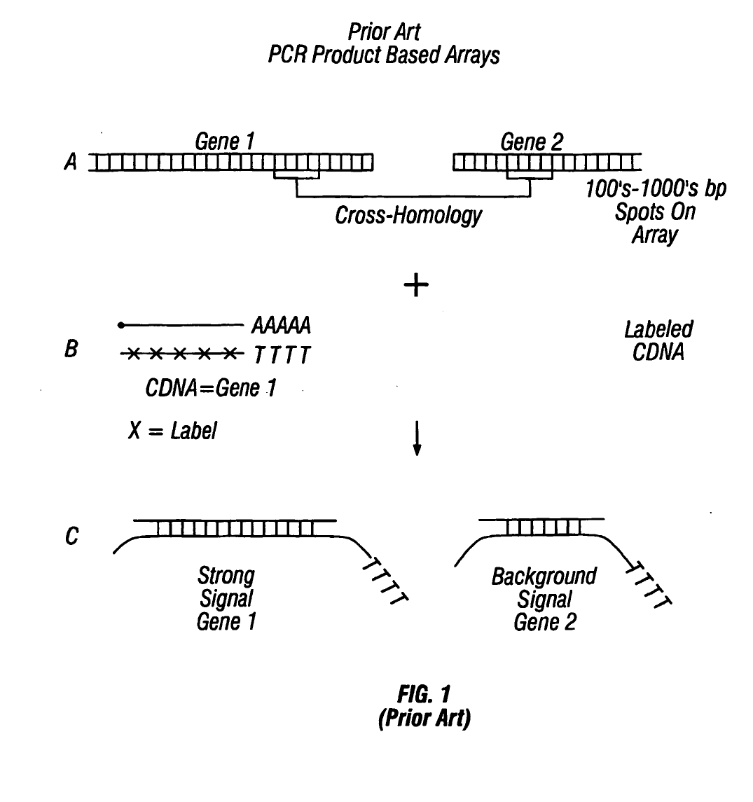

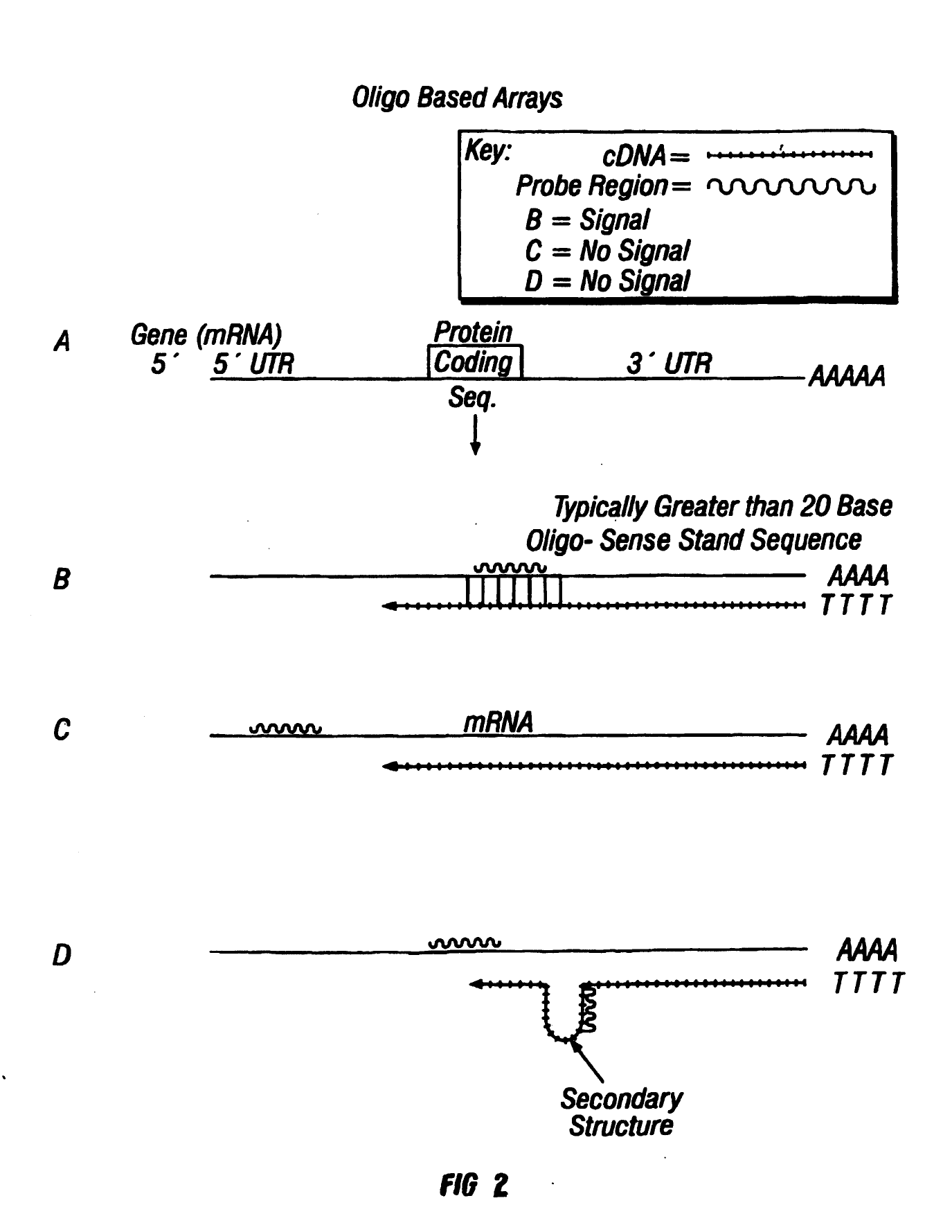

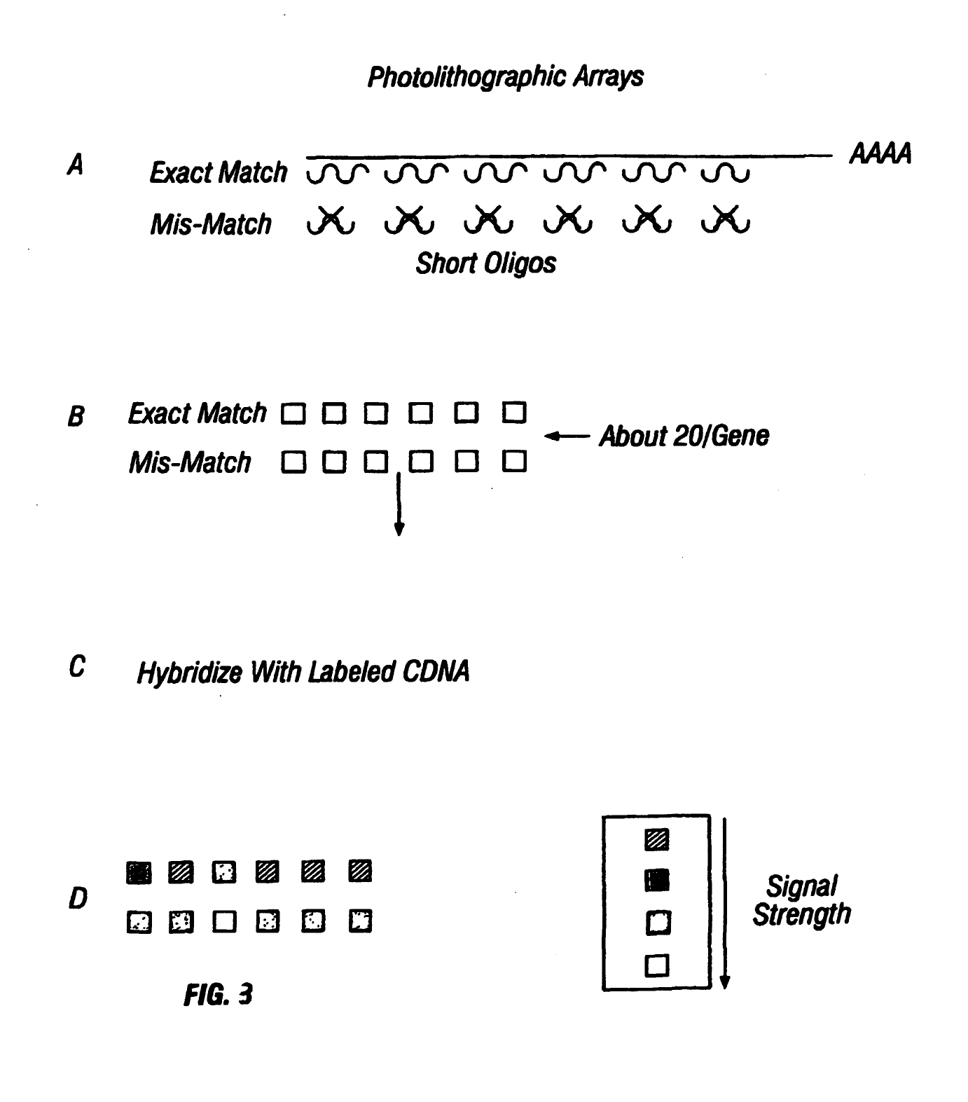

### DETAILED DESCRIPTION

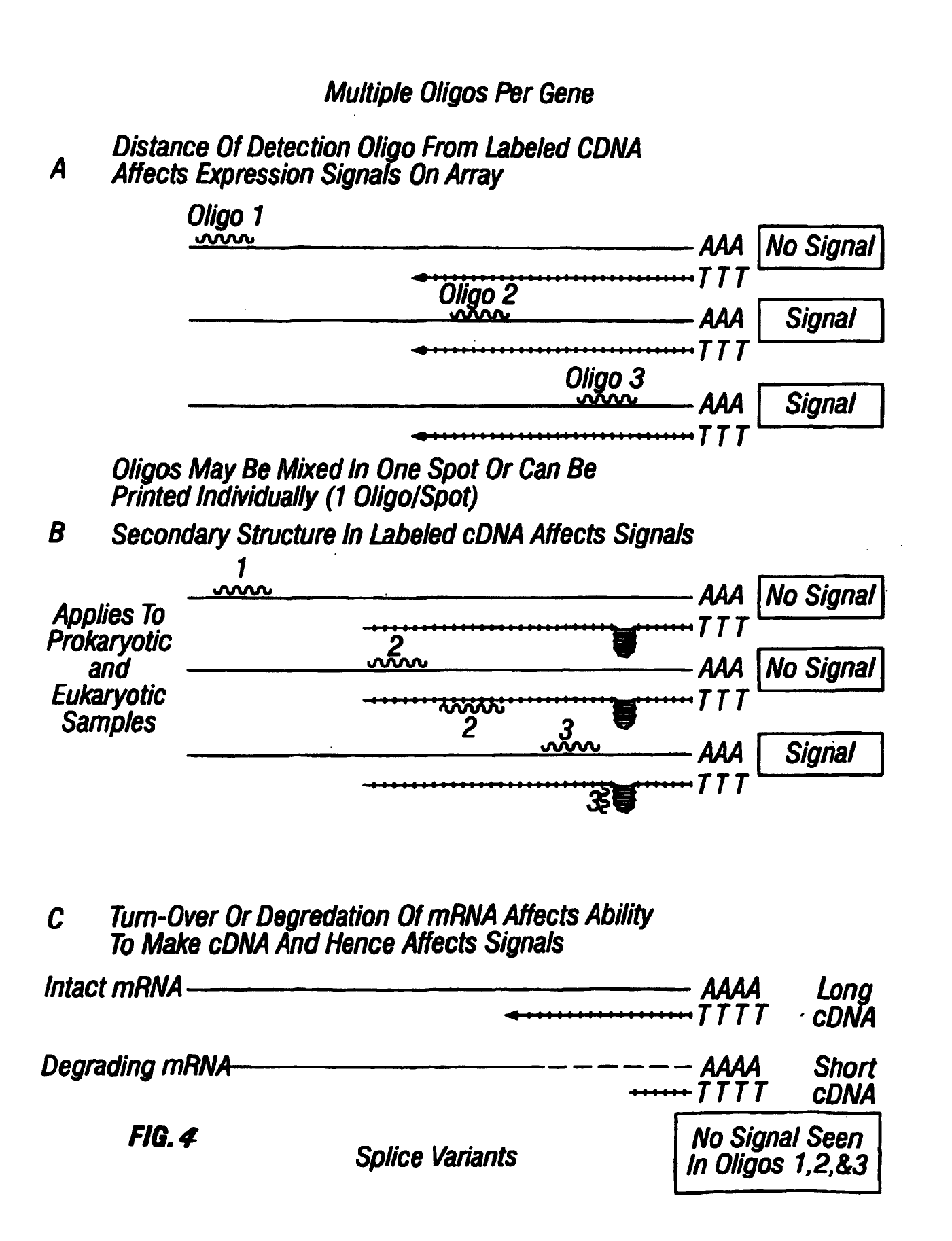

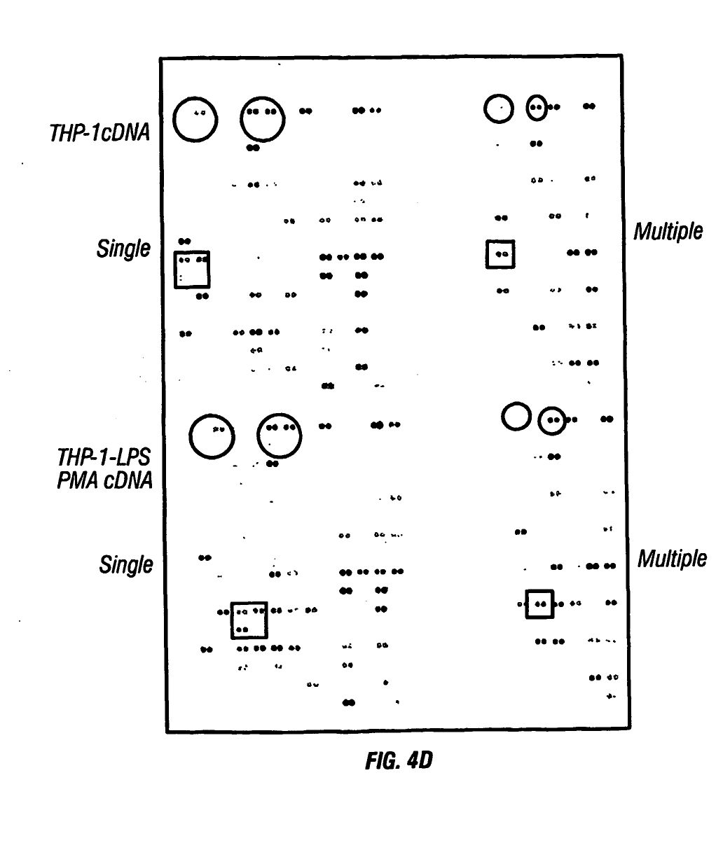

A first embodiment of the invention is described with respect to the use of multiple probes per targeted sequence, preferably at least two probes per target sequence, more preferably three probes per target. FIG. 4 depicts the benefit of using more than one oligonucleotide as detection probes for any individual target sequence. FIG. 4A maps the theoretical location of three distinct oligonucleotides, shown as oscillating lines, with respect to a target sequence polyadenylated mRNAs. The use oligo dT as the labeling primer creates a situation in which the labeled antisense cDNAs, shown as hatched lines, may not be extended to a length sufficient hybridize with the selected oligonucleotide probes. In other words, the distance of a particular detection oligonucleotide probe relative to the extended end of it target cDNA will impact whether that probe will generate a signal positive for expression. For example, in the diagram if “Oligo 1” was printed into an array at a discrete location it would not detect the generated target, while “Oligos 2 and 3” would detect it.

However, the three oligonucleotides, “1, 2 and 3” may be mixed together and printed into one discrete location within the array, thereby decreasing the likelihood of obtaining a false negative result. These probes are of sufficient length in order to decrease non-specific binding, preferably 10 to 200 bases, more preferably 20 to 100 bases, more preferably 40-80 bases. Although “Oligo 1” would not generate a signal, “Oligos 2 and 3” would generate a signal within the same location on the array. In order to facilitate this probe cooperation, the distribution or the placement of the oligonucleotides throughout the target sequence may be deliberately engineered toward any bias known, such that probes may be designed towards the 3′ end, 5′ end and/or center of the target sequence. FIG. 4B depicts the effect secondary structures within the labeled cDNA target may have on signals from multiple oligonucleotide probes. In this example the cDNA target has a large hairpin which masks the sequence which is complementary to “Oligo 3.” However, “Oligo 2” will still generate a signal because its homologous region is within the extension capabilities of the polymerase used, while “Oligo 1” will not contribute to the signal in light of the short length of the labeled cDNA.

FIG. 4C illustrates a further application in which multi-probe cooperation may aid in signal detection. RNA is difficult to manipulate and maintain intact and often degrades prior to use. In fact, between different cell types, different physiological conditions, different RNAs may naturally degrade or “turn-over” at different rates and from different locations within the molecule. Some may preferentially degrade from the 5′ end and others from the 3′ end and still others internally. In addition, patterns of RNA degradation are likely to change within the same system with regard to various populations of cells undergoing differing physiological conditions. Furthermore, for many genes the individual splice variants are unknown and even genes with known splice variants tend to express their different splice variants in a tissue-specific or cell type-specific manner, such that, for example, brain tissue may tend to omit one particular exon in the mature message whereas the spleen might exclude a different exon. Therefore, particular probe designs may target an exon that is actually missing from the message templates and hence cDNA targets of a particular tissue. In many instances none of this information is known going into an expression array experiment, therefore the use of multiple oligonucleotide probes per target sequence per discrete array location may enhance the probability of detecting a signal.

However, an RNA molecule degrading from its 3′ end, as depicted in FIG. 4C by a dashed line, may create a situation in which the use of oligo dT primers to generate a labeled cDNA target will only create a very short labeled product that is incapable of hybridizing to any of the selected oligonucleotide probes. Furthermore, total RNA is predominantly composed of ribosomal RNA, tRNAs, small nuclear RNAs, and the mRNA population within the total pool may only comprise about two to five percent of the entirety. Therefore, mRNA purification using a poly-A purification method followed by an oligo dT labeling, tends to distort the relative proportions of cDNAs within a sample. This is because any method that is used to select a certain species of RNA is likely to have a bias for a particular species of mRNA. Also, not all mRNAs have a poly-A tail, eliminating them from this type of manipulation. Therefore, in some instances it may be desirable to use total RNA as the starting material for the generation of labeled cDNA targets.

These RNA characteristics, degradation potential and structural features, make an alternative priming mechanism for reverse transcription extremely desirable. The use of random primers may avoid many of the problems associated with the loss of template, but their use is not advisable for total RNA as they will generate cDNA molecules from both mRNA and non-mRNA resulting in an increased potential for non-specific background problems.

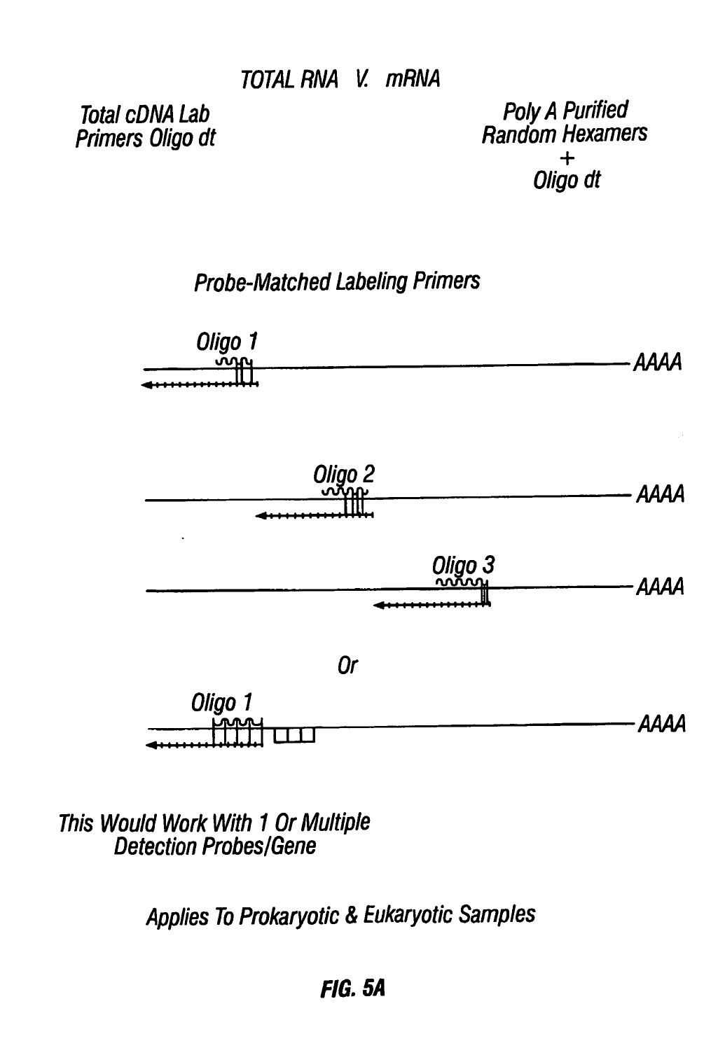

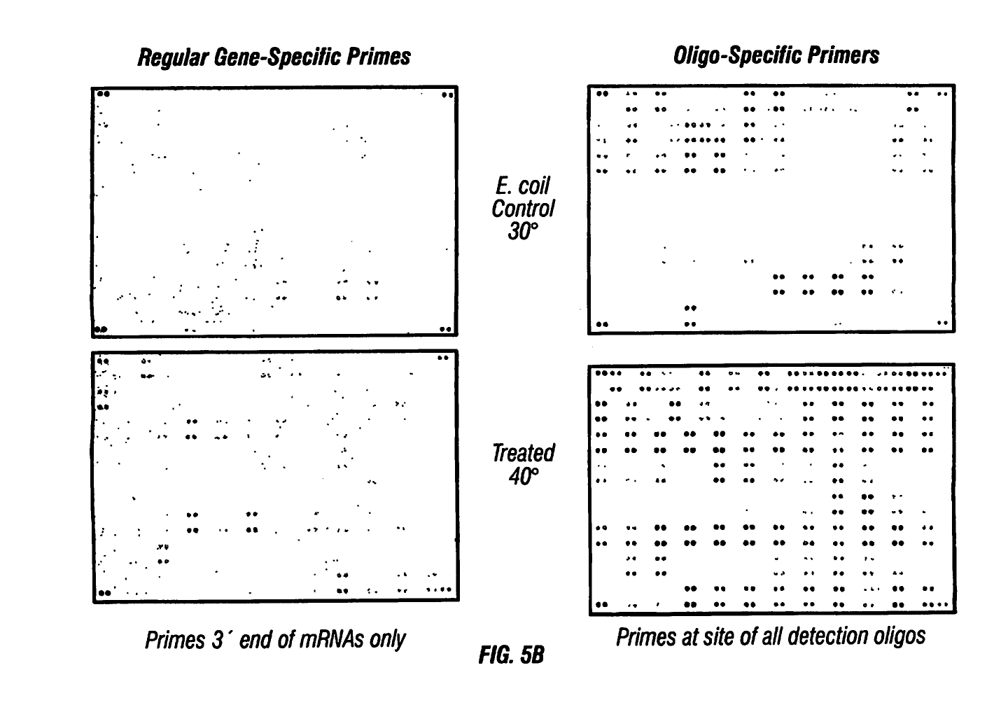

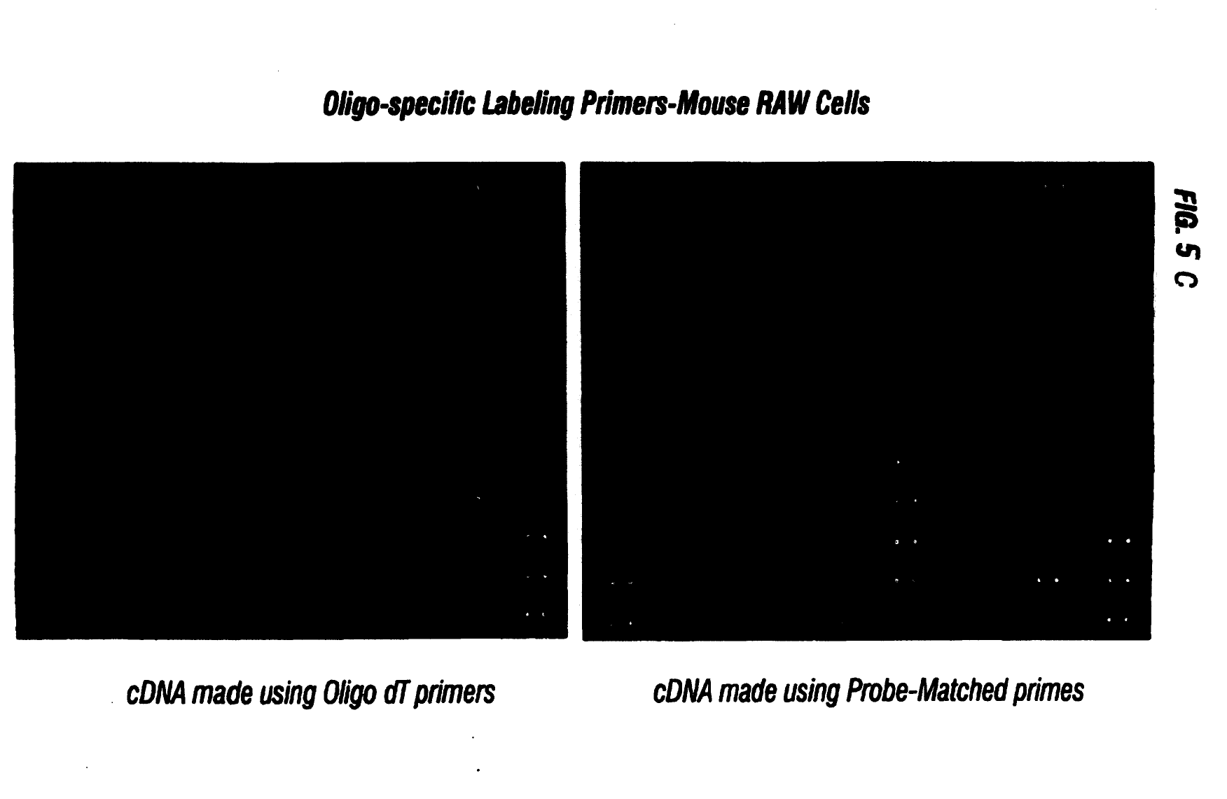

Therefore, according to another aspect, the current invention involves the use of probe matched labeling primers, also referred to in this disclosure as targeted labeling primers or probe matched target primers (PMTPs). FIG. 5 demonstrates the principle of using PMTPs. The probe is the detection oligonucleotide that will be spotted onto the array and the target is the labeled cDNA that is hybridized to the array. In FIG. 5, the line represents a mRNA and the oscillating line depicts the oligonucleotides probes 1, 2 and 3, while the hatched lines indicate the complimentary DNA or cDNA. In this diagram, the labeling primer is actually complimentary to the 3′ end of the detection probe such that any cDNA generated will be complimentary to the oligonucleotide probe. In another preferred embodiment, the primers may be complementary to a portion of a given probe. In other words, the individual primers used are probe matched target primers or PMTPs.

In another embodiment of this invention the individual PMTPs are matched to a region of the target downstream from where the detection probe is homologous to the target. The PMTP may bind to a region of a particular RNA at any extendable distance downstream to where the detection probe is complementary, more preferably 20 to 200 bases downstream, such that any cDNA generated from said labeling primer will be complimentary to the detection probe resulting in a signal if that sequence is truly present. This approach produces significantly stronger signals as compared to oligo dT labeling and because of the sequence specific nature of these PMTPs, they can be used directly with total RNA to generate labeled cDNA targets without incurring the background problems associated with random primers.

Another embodiment of this invention is the use of PMTPs with multiple oligonucleotide probes per discrete location within the array.

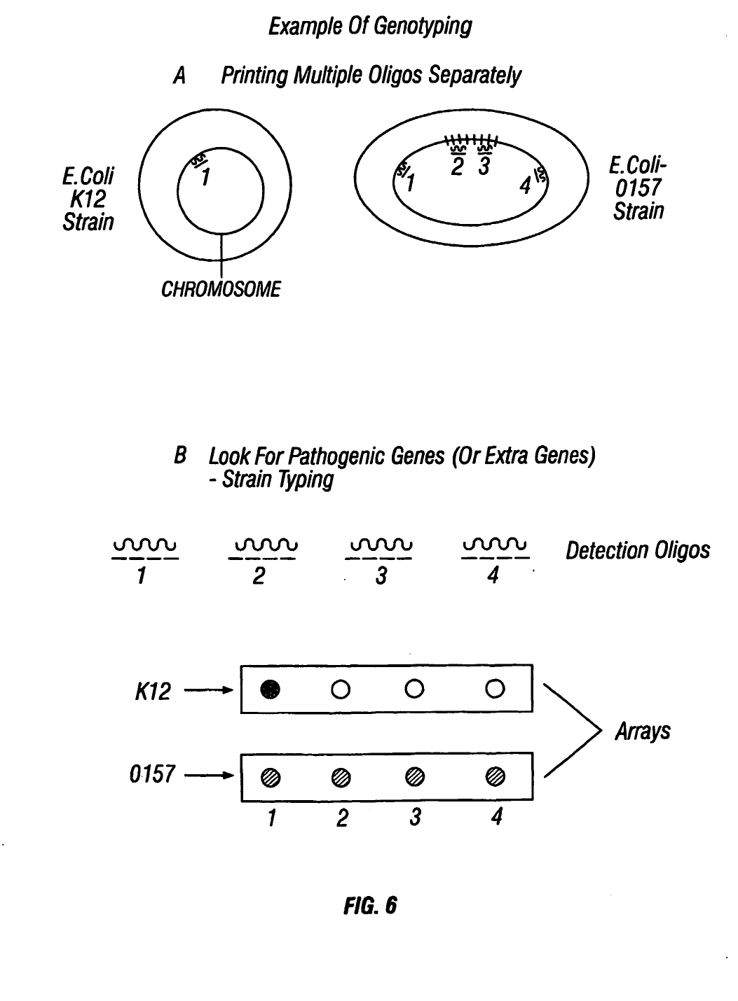

Another embodiment in the use of oligonucleotide arrays and probe matched labeling primers may be for strain typing of bacterial samples, as illustrated in FIG. 6. As shown in FIG. 6, E. coli K12 and E. coli 0157 are separate strains of the same species of bacterium which harbor genetic differences. Oligonucleotide probes are designed to different portions of a genome and probe matched target primers are generated. The target primers may then be hybridized to DNA and a labeling extension reaction may be performed. The resulting labeled DNA may then be hybridized to an array that contains the matched probes. From the data generated, different strains of the same organism can be differentiated.

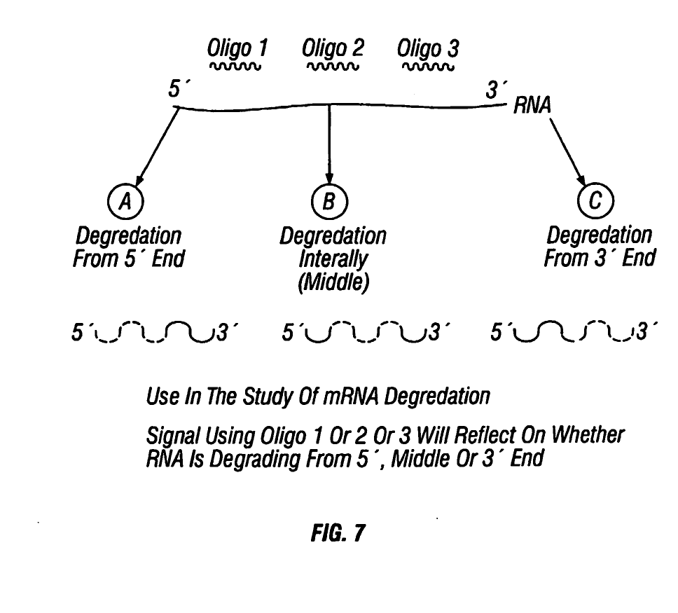

Another embodiment of the invention is to enable the study of differential degradation within a particular RNA. mRNA may degrade from its 5′ end, center, or 3′ end and there are different patterns of degradation among certain populations of cells within the same system. As shown in FIG. 7, oligonucleotide probes and random primers or probe matched primers may bind to an mRNA assuming that the target hybridization sequence is still present. If the target has been degraded, certain probes will most likely no longer bind to it. This can be applied to both eukaryotic and prokaryotic populations of cells. One of many applications of this invention involves studying the turnover rate and/or differential degradation of RNA(s) and still another application involves expression profiling, showing that there is differential expression between a certain set of genes in two different samples.

Another embodiment may be utilization of the invention in genotyping. The specific pattern of matched probe oligonucleotides to targets may detail the following: presence and/or absence of point mutations and amplification and/or deletions of certain sequences. This encompasses SNP analysis, fingerprinting, mutation detection, and other genetic assays.

Another embodiment is useful for a variation of comparative genomic hybridization (CGH). CGH may be used for analyzing gross differences between two biological samples. For example, solid tumors tend to show amplification and/or deletions of chromosome base pairs or chromosomal domains. By labeling two different samples with two different colored fluorescent dyes and hybridizing the mixture to a chromosomal preparation, these regional copy number differences can be noted. In the same respect, probes can be designed to different portions of the genome at desired spacing in order to finely map or grossly map the presence or absence and/or copy number of chromosomal regions between two different samples.

Both genotyping and comparative genomic hybridization may be accomplished with PMTP oligonucleotide arrays. The target needs to be only long enough for probe(s) to bind to it. Any genomic sample may be labeled and hybridized to the array. Patterns of signals from the arrays may then be analyzed and characterized with regards to a particular organism or strain.

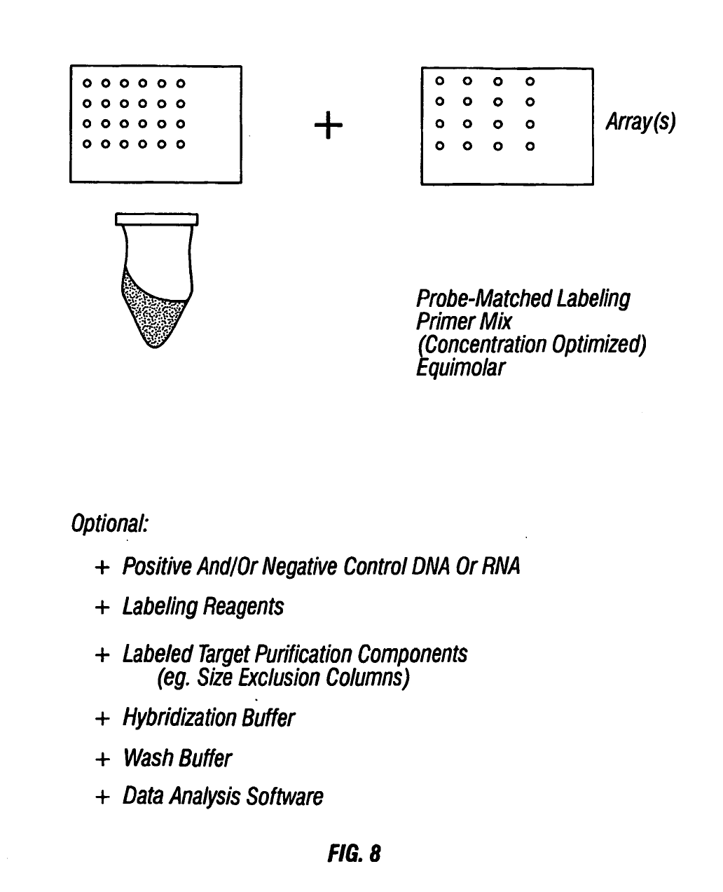

In another aspect, the current invention provides a kit. Each kit contains an array providing multiple oligonucleotides probes per gene, according to the inventive “multiple oligo” method described above. The kit further contains PMTPs, according to the inventive “PMTP” method also described above. This is detailed in FIG. 8. The kit advantageously contains the probe matched labeling primer mix in a concentration optimized to work in the application for which it is designed. Optional components also provided with the kit might include positive and/or negative control DNA or RNA, labeling reagents, non-labeled probes, polymerase reaction buffer, labeled target purification components, hybridization buffers, wash buffers, and other necessary signal detection components, including analysis software.

Another embodiment is utilization of the probe-matched primers, nucleotides, and appropriate enzymes to render the immobilized probes as double-stranded DNA molecules. The resulting double-stranded DNA probes may be used as substrates for detecting the presence or absence of sequence-specific DNA-binding interactions. These include but are not limited to assays to detect of protein-DNA interactions and drug-DNA interactions.

Another embodiment is utilization of the probe-matched primers, labeled nucleotides, and appropriate enzymes to render the immobilized probes as labeled double-stranded DNA molecules. The resulting double-stranded DNA probes could be used as substrates for detecting the presence or absence of sequence-specific DNA-binding interactions. These include but are not limited to assays to detect enzyme-DNA interactions.

It will be appreciated that such a kit is a convenient commercial embodiment enabling users to practice the above described “multiple oligo” invention and/or the above described “PMTP” invention. Kits may be packaged so as to allow users to conduct separate assays in identifying and classifying genetic material.

Although the present invention and its advantages have been described in detail, it should be understood that various changes, substitutions and alterations can be made herein without departing from the spirit and scope of the invention as defined by the appended claims.

1. A method for nucleic acid array design, the array containing individual nucleic acid sequences or portions thereof, the method comprising:
(a) providing at least two discrete oligos per target nucleic acid sequence or portions thereof;
(b) printing the at least two discrete oligos onto a distinct location within the array; and
(c) using the array in genetic analysis.

2. The method of claim 1, wherein (a) includes providing three discrete oligos per target nucleic acid sequence or portions thereof.

3. The method of claim 1, wherein (a) includes providing the at least two discrete oligos which range from 4 to 120 nucleotides in length.

4. The method of claim 1, wherein (a) includes providing the at least two discrete oligos which range from 20 to 80 nucleotides in length.

5. The method of claim 1, wherein (c) includes generating labeled nucleic acids from a sample nucleic acid population using a probe matched target primer.

6. The method of claim 1, wherein (a) includes providing the at least two discrete oligos which are complementary to non-contiguous sections of the target nucleic acid sequence.

7. The method of claim 1, wherein (a) includes providing the at least two discrete oligos which are complementary to overlapping sections of the target nucleic acid sequence.

8. The method of claim 1, wherein (a) includes providing the at least two discrete oligos which are complementary to partially overlapping sections of the target nucleic acid sequence.

9. The method of claim 1, wherein (a) includes providing the at least two discrete oligos which recognize non-contiguous sections of the target nucleic acid sequence.

10. The method of claim 1, wherein (a) includes providing the at least two discrete oligos which recognize overlapping sections of the target nucleic acid sequence.

11. The method of claim 1, wherein (a) includes providing the at least two discrete oligos which recognize partially overlapping sections of the target nucleic acid sequence.

12. The method of claim 1, wherein (a) includes providing the at least two discrete oligos which are complementary to more than one allele for a given locus.

13. The method of claim 1, wherein (a) includes providing the at least two discrete oligos which are complementary to more than one splice variant of a given mRNA.

14. The method of claim 1, wherein (a) includes providing the at least two discrete oligos which recognize a specific functional class of genes.

15. The method of claim 1, wherein (a) includes providing the at least two discrete oligos which recognize a specific domain which is conserved among a particular class of genes.

16. The method of claim 1, wherein (a) includes providing the at least two discrete oligos which recognize a specific domain which is unique among a particular class of genes.

17. The method of claim 1, wherein (a) includes providing the at least two discrete oligos that are not complementary to a target nucleic acid sequence.

18. The method of claim 1, wherein (a) includes providing the at least two discrete oligos that contain a randomized sequence.

19. The method of claim 1, wherein (a) includes providing the at least two discrete oligos that are complementary to a house-keeping gene.

20. The method of claim 1, wherein (a) includes providing the at least two discrete oligos that are complementary to a tRNA gene.

21. The method of claim 1, wherein (a) includes providing the at least two discrete oligos that are complementary to a ribosomal gene.

22. The method of claim 1, wherein (a) includes providing the at least two discrete oligos that are complementary to a heterologous gene to be used as a normalization control for the array.

23. The method of claim 1, wherein (b) includes printing between 100 and 5000 distinct locations within the array.

24. An array comprising at least two discrete locations, wherein each discrete location comprises at least two distinct oligos per target nucleic acid sequence or portions thereof.

25. The array of claim 24, comprising between 100 and 5000 discrete locations within the array.

26. The array of claim 24, wherein the at least two distinct oligos comprises three discrete oligos per target nucleic acid sequence or portions thereof.

27. The array of claim 24, wherein the at least two distinct oligos per target nucleic acid sequence or portions thereof, comprises distinct oligos which range from 4 to 120 nucleotides in length.

28. The array of claim 24, wherein the at least two distinct oligos per target nucleic acid sequence or portions thereof, comprises distinct oligos which range from 20 to 80 nucleotides in length.

29. The array of claim 24, wherein the at least two discrete oligos are complementary to non-contiguous sections of the target nucleic acid sequence.

30. The array of claim 24, wherein the at least two discrete oligos are complementary to overlapping sections of the target nucleic acid sequence.

31. The array of claim 24, wherein the at least two discrete oligos are complementary to partially overlapping sections of the target nucleic acid sequence.

32. The array of claim 24, wherein the at least two discrete oligos recognize non-contiguous sections of the target nucleic acid sequence.

33. The array of claim 24, wherein the at least two discrete oligos recognize overlapping sections of the target nucleic acid sequence.

34. The array of claim 24, wherein the at least two discrete oligos recognize partially overlapping sections of the target nucleic acid sequence.

35. The array of claim 24, wherein the at least two discrete oligos are complementary to more than one allele for a given locus.

36. The array of claim 24, wherein the at least two discrete oligos are complementary to more than one splice variant of a given RNA.

37. The array of claim 24, wherein the at least two discrete oligos recognize a specific functional class of genes.

38. The array of claim 24, wherein the at least two discrete oligos recognize a specific domain which is conserved among a particular class of genes.

39. The array of claim 24, wherein the at least two discrete oligos recognize a specific domain which is unique among a particular class of genes.

40. A method of use for an array, comprising:
(a) providing an array comprising at least two discrete locations, wherein each discrete location comprises at least two distinct oligos per target nucleic acid sequence or portions thereof;
(b) generating a labeled nucleic acid from a sample nucleic acid population using a probe matched target primer; and
(c) hybridizing the labeled nucleic acids to the array.

41. The method of claim 40, wherein the probe matched target primer binds to a region in the sample nucleic acid population that is in the range of 15 to 100 nucleotides downstream of the region to which at least one of the at least two distinct oligos is complementary.

42. The method of claim 40, wherein the probe matched target primer binds to a region in the sample nucleic acid population that is contiguous with the region to which at least one of the at least two distinct oligos is complementary.

43. The method of claim 40, wherein the probe matched target primer binds to a region in the sample nucleic acid population that overlaps the region to which at least one of the at least two distinct oligos is complementary.

44. The method of claim 40, wherein the probe matched target primer binds to a region in the sample nucleic acid population that partially overlaps the region to which at least one of the at least two distinct oligos is complementary.

45. The method of claim 40, wherein the at least two distinct oligos per target nucleic acid sequence or portions thereof, range from 4 to 120 nucleotides in length.

46. The method of claim 40, wherein the at least two distinct oligos per target nucleic acid sequence or portions thereof, range from 20 to 80 nucleotides in length.

47. The method of claim 40, wherein the at least two distinct oligos per target nucleic acid sequence or portions thereof, are complementary to non-contiguous sections of the target nucleic acid sequence.

48. The method of claim 40, wherein the at least two distinct oligos per target nucleic acid sequence or portions thereof, are complementary to overlapping sections of the target nucleic acid sequence.

49. The method of claim 40, wherein the at least two distinct oligos per target nucleic acid sequence or portions thereof, are complementary to partially overlapping sections of the target nucleic acid sequence.

50. The method of claim 40, wherein the at least two distinct oligos per target nucleic acid sequence or portions thereof, recognize non-contiguous sections of the target nucleic acid sequence.

51. The method of claim 40, wherein the at least two distinct oligos per target nucleic acid sequence or portions thereof, recognize overlapping sections of the target nucleic acid sequence.

52. The method of claim 40, wherein the at least two distinct oligos per target nucleic acid sequence or portions thereof, recognize partially overlapping sections of the target nucleic acid sequence.

53. The method of claim 40, wherein the at least two distinct oligos per target nucleic acid sequence or portions thereof, are complementary to more than one allele for a given locus.

54. The method of claim 40, wherein the at least two distinct oligos per target nucleic acid sequence or portions thereof, are complementary to more than one splice variant of a given mRNA.

55. The method of claim 40, wherein the at least two distinct oligos per target nucleic acid sequence or portions thereof, recognize a specific functional class of genes.

56. The method of claim 40, wherein the at least two distinct oligos per target nucleic acid sequence or portions thereof, recognize a specific domain which is conserved among a particular class of genes.

57. The method of claim 40, wherein the at least two distinct oligos per target nucleic acid sequence or portions thereof, recognize a specific domain which is unique among a particular class of genes.

58. A method for genetic analysis, comprising:
(a) generating a labeled nucleic acid from a sample nucleic acid population using a probe matched target primer;
(b) hybridizing the labeled nucleic acid to an array; and
(c) analyzing the array.

59. The method of claim 58, wherein the labeled nucleic acid is labeled with a fluorescent dye.

60. The method of claim 58, wherein the labeled nucleic acid is labeled with a protein moiety.

61. The method of claim 58, wherein the labeled nucleic acid is labeled with a radioactive label.

62. The method of claim 58, wherein the labeled nucleic acid is labeled with a lamthanide.

63. The method of claim 58, further comprising a wash step after step (b).

64. The method of claim 58, wherein the probe matched target primer binds to a region in the sample nucleic acid population that is in the range of 15 to 100 nucleotides downstream of the region to which at least one of the at least two distinct oligos is complementary.

65. The method of claim 58, wherein the probe matched target primer binds to a region in the sample nucleic acid population that is contiguous with the region to which at least one of the at least two distinct oligos is complementary.

66. The method of claim 58, wherein the probe matched target primer binds to a region in the sample nucleic acid population that overlaps the region to which at least one of the at least two distinct oligos is complementary.

67. The method of claim 58, wherein the probe matched target primer binds to a region in the sample nucleic acid population that partially overlaps the region to which at least one of the at least two distinct oligos is complementary.

68. The method of claim 58, wherein step (c) comprises the use of a phosphoimager.

69. The method of claim 58, wherein step (c) comprises the use of electroluminescence.

70. The method of claim 58, wherein step (c) comprises the use of a laser.

71. A kit having component parts capable of being used in combination for testing genetic material for the presence of predetermined nucleic acid sequences, the kit comprising the combination of:
an array containing at least one discrete oligo per nucleic acid sequence or portions thereof, and
at least one probe matched target primer.

72. The kit of claim 71, wherein the at least one discrete oligo per nucleic acid sequence or portions thereof, comprises at least three discrete oligos.

73. The kit of claim 71, wherein the at least one discrete oligos per target nucleic acid sequence or portions thereof, comprises discrete oligos which range from 4 to 120 nucleotides in length.

74. The kit of claim 71, wherein the at least one discrete oligos per target nucleic acid sequence or portions thereof, comprises discrete oligos which range from 20 to 80 nucleotides in length.

75. The kit of claim 71, wherein the at least one discrete oligo per nucleic acid sequence or portions thereof, comprises at least two discrete oligos.

76. The kit of claim 75, wherein the at least two discrete oligos are complementary to non-contiguous sections of the target nucleic acid sequence.

77. The kit of claim 75, wherein the at least two discrete oligos are complementary to overlapping sections of the target nucleic acid sequence.

78. The kit of claim 75, wherein the at least two discrete oligos are complementary to partially overlapping sections of the target nucleic acid sequence.

79. The kit of claim 75, wherein the at least two discrete oligos recognize non-contiguous sections of the target nucleic acid sequence.

80. The kit of claim 75, wherein the at least two discrete oligos recognize overlapping sections of the target nucleic acid sequence.

81. The kit of claim 75, wherein the at least two discrete oligos recognize partially overlapping sections of the target nucleic acid sequence.

82. The kit of claim 75, wherein the at least two discrete oligos are complementary to more than one allele for a given locus.

83. The kit of claim 75, wherein the at least two discrete oligos are complementary to more than one splice variant of a given RNA.

84. The kit of claim 75, wherein the at least two discrete oligos recognize a specific functional class of genes.

85. The kit of claim 75, wherein the at least two discrete oligos recognize a specific domain which is conserved among a particular class of genes.

86. The kit of claim 75, wherein the at least two discrete oligos recognize a specific domain which is unique among a particular class of genes.

87. The kit of claim 71, wherein the probe matched target primer binds to a region in the sample nucleic acid population that is in the range of 15 to 100 nucleotides downstream of the region to which at least one of the at least one distinct oligos is complementary.

88. The kit of claim 71, wherein the probe matched target primer binds to a region in the sample nucleic acid population that is contiguous with the region to which at least one of the at least one distinct oligos is complementary.

89. The kit of claim 71, wherein the probe matched target primer binds to a region in the sample nucleic acid population that overlaps the region to which at least one of the at least one distinct oligos is complementary.

90. The kit of claim 71, wherein the probe matched target primer binds to a region in the sample nucleic acid population that partially overlaps the region to which one of the at least one distinct oligos is complementary.

91. The kit of claim 71, further comprising a wash buffer.

92. The kit of claim 71, further comprising a hybridization buffer.

93. The kit of claim 71, further comprising a labeling reagent.

94. The kit of claim 71, further comprising a positive control.

95. The kit of claim 71, further comprising a negative control.
